(19) United States
(12) Patent Application Publication
(10) Pub. No.: US 2013/0005001 A1
(43) Pub. Date: Jan. 3, 2013

(54) METHOD FOR PRODUCING NEBIVOLOL

(75) Inventors: Gerhard Jas, Berlin (DE); Ilia Freifeld, Frankfurt/Main (DE); Kurt Kesseler, Hofheim (DE)

(73) Assignee: CORDEN PHARMA INTERNATIONAL GMBH

(21) Appl. No.: 13/574,348

(22) PCT Filed: Jan. 21, 2011
(86) PCT No.: PCT/EP2011/000246

§ 371 Date: Aug. 20, 2012

(30) Foreign Application Priority Data
Jan. 27, 2010 (DE) 10 2010 005 953.6

(51) Int. Cl. C07D 407/12; C12P 17/16

(52) U.S. Cl. 435/118; 549/407

(57) ABSTRACT

The present invention relates to a method for producing racemic nebivolol represented by general formula (I)

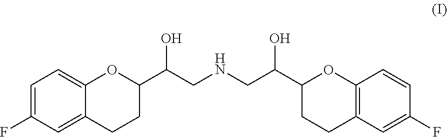

from the enantiomerically-pure compounds represented by formula (IVa) and (IVb);

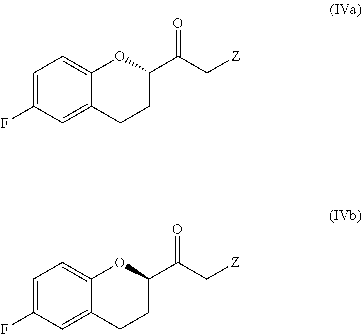

whereby racemic nebivolol is obtained through mixing enantiomerically-pure d-nebivolol and l-nebivolol which are synthesised independent of each other as enantiomerically-pure compounds through individual coupling of the 4 enantiomerically-pure key intermediates represented by formula (IIa-d) to the corresponding precursors represented by formula (IIIa-d);

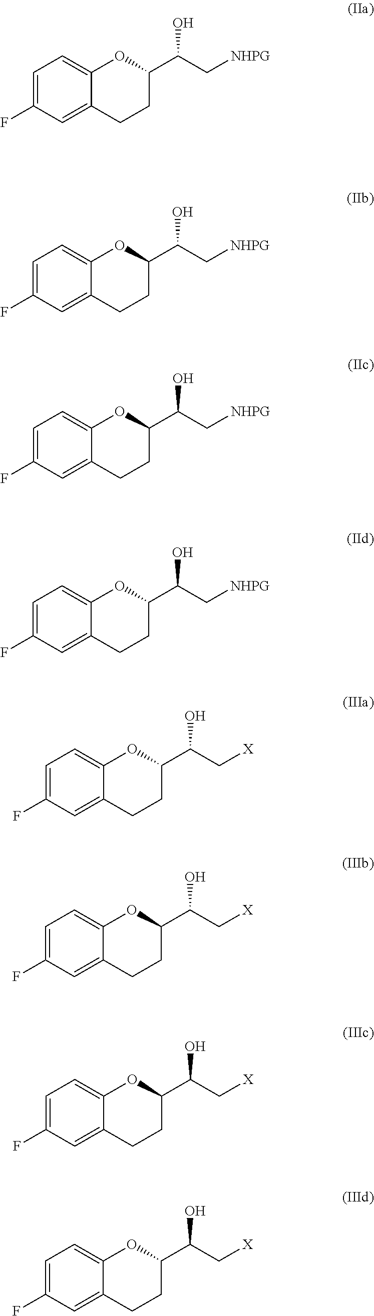

whereby d-nebivolol (Ia) is obtained through coupling (IIa) to (IIIb) or (IIb) to (IIIa) and l-nebivolol (Ib) is obtained through coupling (IIc) to (IIId) or (IId) to (IIIc), and PG in the intermediates represented by formula (IIa-d) is a hydrogen atom or an amine protection group, and X in the precursors represented by formula (IIIa-d) is a halogen atom, a hydroxyl group, an acyl group, an alkylsulfonyloxy group or an arylsulfonyloxy group, whereby intermediate (IIa) is formed from (IIIa), intermediate (IIb) is formed from (IIIb), intermediate (IIc) is formed from (IIIc), and intermediate (IId) is formed from (IIId), whereby the precursors represented by formula (IIIa) and (IIId) originate from the ketone precursor represented by formula (IVa), and the precursors represented by formula (IIIb) and (IIIc) originate from the ketone precursor represented by formula (IVb), and Z in the ketone precursors (IVa,b) is a halogen atom, a hydroxyl function, an acyl group, an alkylsulfonyloxy group or an arylsulfonyloxy group.

The present invention describes a new method for producing racemic nebivolol of general formula (I).

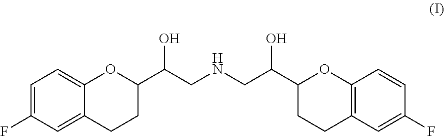

Nebivolol is an active substance that belongs to the group of selective β₁-adrenoreceptor blockers (R-blockers) and is used to treat high blood pressure. Nebivolol is applied in the form of the racemate and consists of two enantiomers, [2S [2R[R [R]]]]α,α′-[imino-bis[methylen]]bis[6-fluoro-chroman-2-methanol] (called d-nebivolol hereinafter) represented by structural formula (Ia) and the [2R [2S[S [S]]]] enantiomer represented by structural formula (Ib) (called l-nebivolol hereinafter).

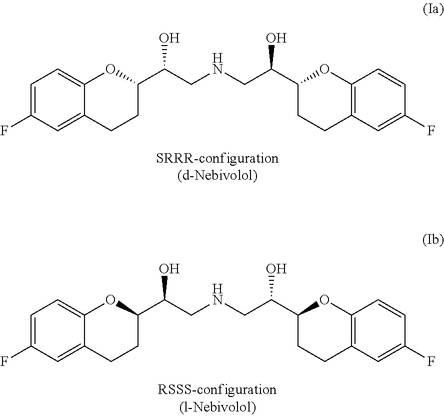 [SMILES: O[C@H](CNC[C@@H](O)[C@H]1CCc2cc(F)ccc2O1)[C@@H]1CCc2cc(F)ccc2O1 ; O[C@@H](CNC[C@H](O)[C@H]1CCc2cc(F)ccc2O1)[C@@H]1CCc2cc(F)ccc2O1]

Owing to its basic properties, nebivolol can be converted into pharmaceutically active acid addition salts, whereby the hydrochloride is the salt that is both commercially available and approved for treatment.

The challenge presented by the synthesis of nebivolol that is immediately obvious to a person skilled in the art are the 4 centres of asymmetry of the target compound which theoretically give rise to 16 conceivable enantiomers. This number is reduced to only 10 possible enantioners owing to the symmetry properties of the compound.

It is not surprising then that the complexity of the structure has led to a multitude of methods proposing a manageable solution. Most of said methods are inconvenient and/or too expensive owing to their lack of stereochemical selection and the resulting laborious separation and cleaning processes of the intermediary diastereomer mixtures that are obtained.

A non-stereoselective synthesis of nebivolol was first described by Janssen Pharmaceutica N.V. in the application EP 0145067 (U.S. Pat. No. 4,654,362) starting with 6-fluoro-4-oxo-4H-1-benzopyran-2-carboxylic acid. The starting point is racemic 6-fluoro-chromancarboxylic acid ethylester, which is then converted into the corresponding aldehyde via a reduction-oxidation sequence. Said aldehyde is converted into a mixture of 4 stereoisomeric epoxides [(R,S)—, (S,R)—, (R,R)—, and (S,S) configuration] using common methods (diagram 1). Said mixture of stereoisomers is then separated by means of chromatography to produce a mixture of the anti-configuration (R,S), (S,R) epoxides and/or syn-configuration (S,S), (R,R) epoxides, which are the central intermediates for further synthesis.

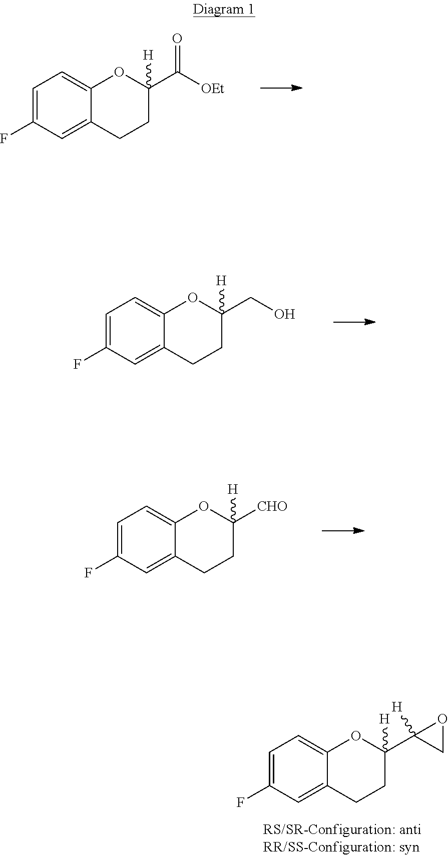 [SMILES: CCOC(=O)C1CCc2cc(F)ccc2O1 ; OCC1CCc2cc(F)ccc2O1 ; O=CC1CCc2cc(F)ccc2O1 ; Fc1ccc2c(c1)CCC(C1CO1)O2]

As a continuation of said approach, application no. EP 0334429 (U.S. Pat. No. 6,545,040) describes a stereoselective synthesis of nebivolol through the selective preparation of d- and/or l-nebivol. This starts with resolving the racemate of 6-fluoro-chromancarboxylic acid by means of (+) dehydroabiethylamine into the two enantiomers, which are then converted into the epoxides through standard steps (diagram 2).

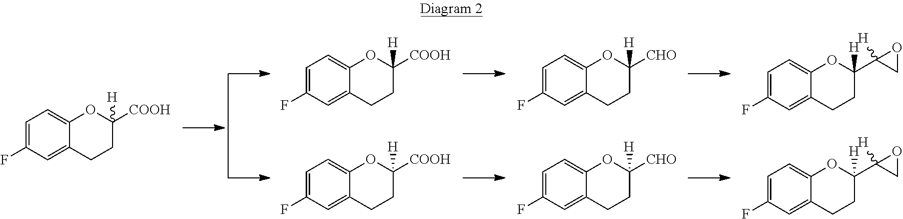

Cimex Pharma AG and University of Zurich describe alternative methods for the preparation of racemic nebivolol and the enantiomers thereof in applications EP 1803715 and EP 1803716. Said methods start from the racemic compound represented by formula (A), where LG=Br or Cl, which is produced according to a new method. Compound (A) is transformed by stereoselective reduction into the alcohols represented by formula (B) from which a mixture of the epoxides (C) is generated.

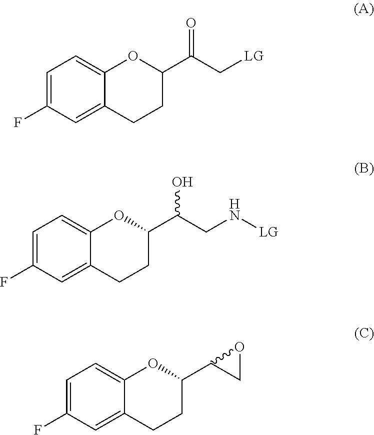

The full synthetic sequence is shown in diagram 3.

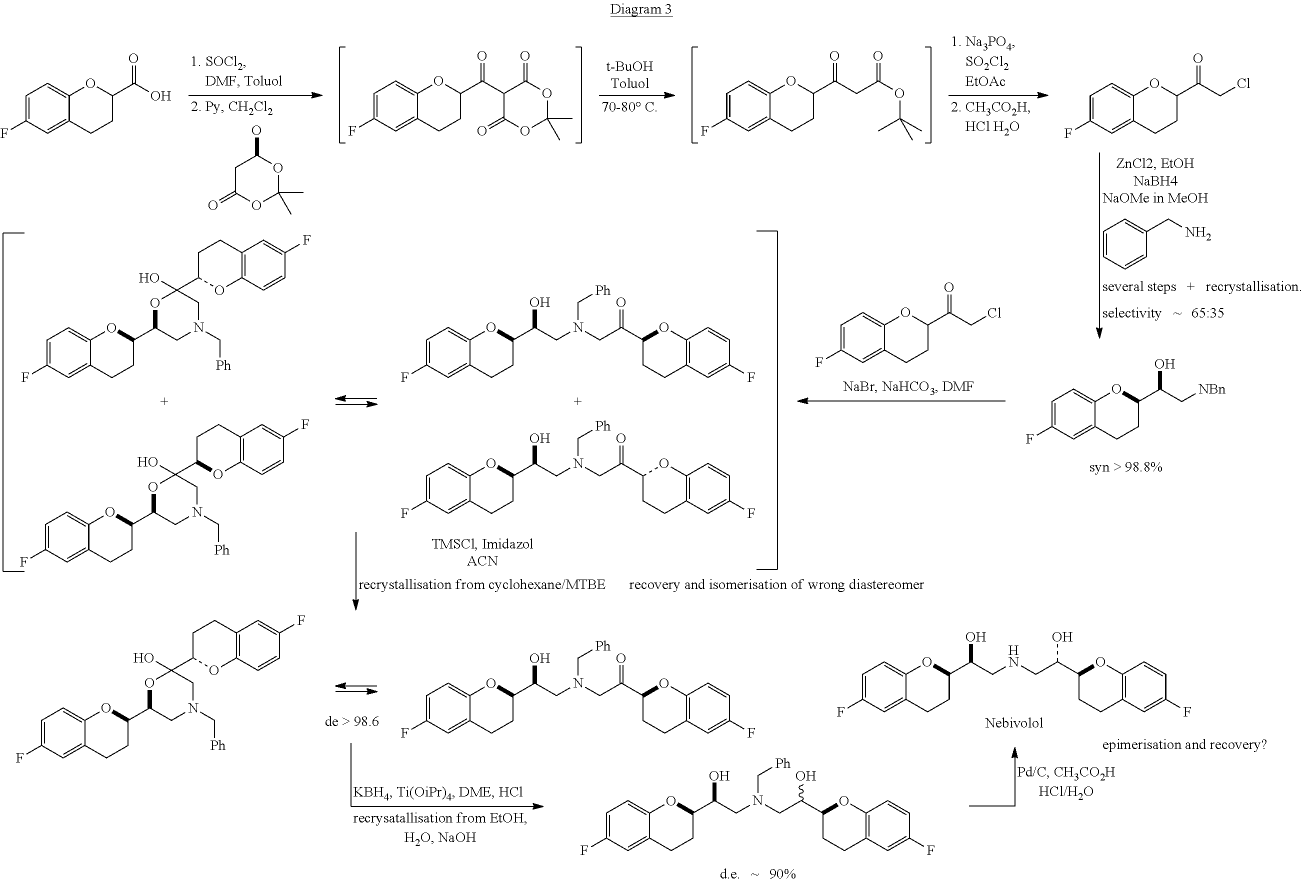

Although all reduction steps in the scope of this method have been investigated and optimised extensively, the overall synthesis ails due to the only moderate selectivities which necessitate laborious crystallisation and recovery steps.

US 2008/0221340 (Pharmacon Forschung and Beratung GmbH) and the corresponding applications EP 1919888 and WO 2007/009143, respectively, describe a process for the preparation of racemic nebivolol that provides for the utilisation of diastereomeric cyanohydrins represented by formula (D), which are produced from racemic 6-fluoro-3,4-dihydro-2H-[1]benzopyran-2-carbaldehyde.

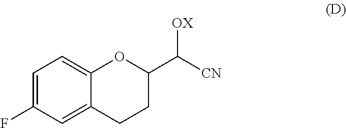

However, the further synthesis entails the use of lithiumaluminiumhydride LiALH₄ as reduction agent for the preparation of the amines from the nitriles as well as of sodium cyanoborohydride in a reductive amination. Both reagents are not amongst the preferred reagents for industrial production. Moreover, a series of fractionated crystallisation steps with subsequent recrystallisation is required to separate the diastereomers in the scope of the synthesis to a sufficient extent to have suitable coupling precursors for racemic nebivolol available. It is questionable if this can be developed into a commercially attractive method.

The recently published application, WO 2009/121710 (ZACH SYSTEM S.P.A.), describes a process for preparation of nebivolol that utilises a haloketone represented by formula (E), which is being reduced stereoselectively by means of (+) or (−) B-chlorodiisopinacampheylborane (DIP chloride) to form diastereomeric alcohols represented by formula (F) which function as intermediates in the production of nebivolol.

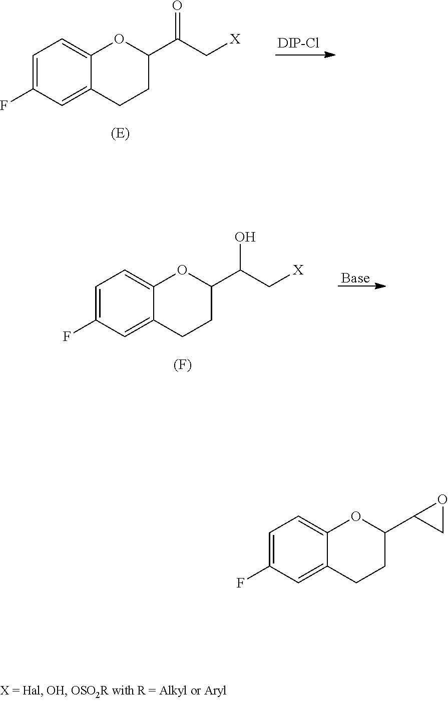

It is reported that the reduction by means of DIP-CI proceeds with good stereoselectivity (approx. 99%). However, this concept also leads to the formation of mixtures of diastereomers in (F) that necessitate subsequent separation, such as, e.g., through hydrolytic kinetic racemate resolution of the type that is described in WO 2008/040528.

Further methods for preparation of nebivolol are claimed in applications WO 2006/025070 (Torrent Pharmaceuticals), WO 2006/016376, and WO 2007/083318 (Hetero Drugs). However, these applications only describe mixtures of diastereomeric intermediates which ultimately necessitate chromatographic separations.

WO 2004/041805 (EGIS GYOGYSZERGYAR) describes the synthesis of 4 enantiomerically-pure intermediates that are converted into two enantiomerically-pure precursors of d- and/or l-nebivolol through suitable coupling processes. Further chemical manipulation of a racemic mixture of said precursors ultimately results in racemic nebivolol. The use of derivatives of D- and/or L-glycerolaldehyde from the chiral pool and separate preparation of the diastereomeric intermediates (G a,b) provides the foundation for the subsequent preparation of the 4 enantiomerically-pure epoxides (H a-d) through standard manipulations such as hydrolysis of the acetal followed by tosylation of the primary hydroxyl function and final cyclisation to form the above-mentioned epoxides (diagram 4). The users claim to be able to attain the requisite separation of the mixtures of diastereomers obtained in the beginning through selective crystallisation methods alone.

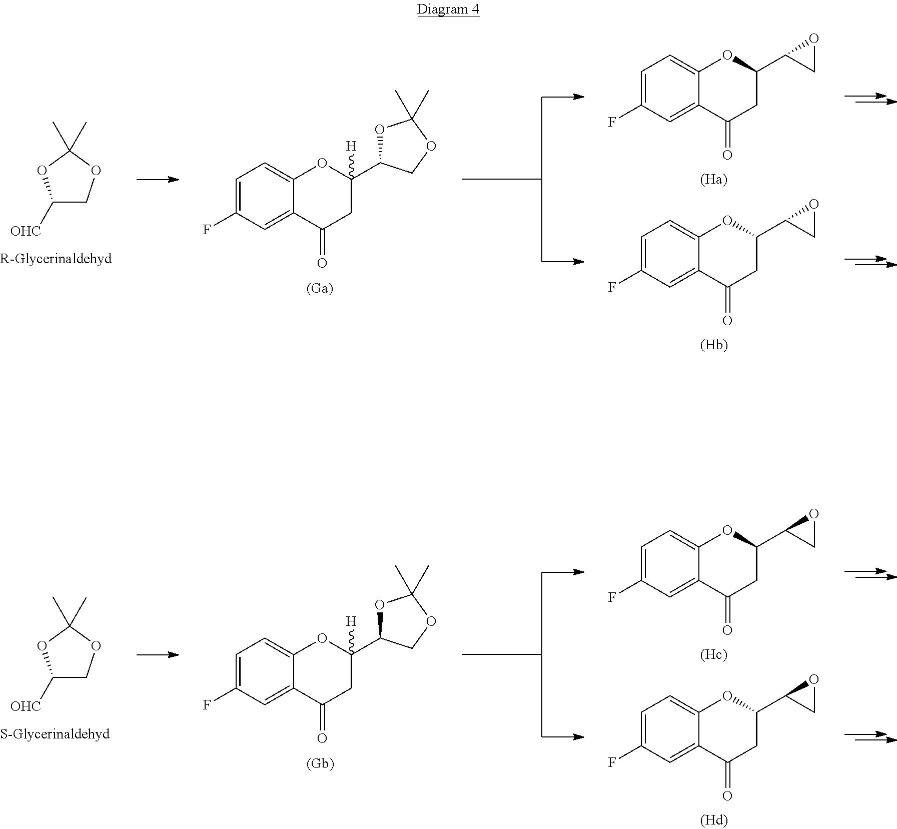

The literature includes some proposals (e.g., B. S. Chandrasekhar, V. Reddy: Tetrahedron 56 (2000), 6339-6344; C. W: Johannes, M. S: Visser, G. S: Weatherhead, A. J. Hoveyda: J. Am. Chem. Soc. 120 (1988), 8340-8347) that are related to the synthesis of pure enantiomers of nebivolol. However, the transfer of said syntheses to industrial scale is hardly feasible for cost reasons.

It is therefore the object of the present invention to provide for a simpler and/or less expensive production of racemic nebivolol.

Surprisingly, said object is solved through the independent synthesis of d- and/or l-nebivolol (Ia) and/or (Ib) as outlined in diagram 5. In this context, enantiomerically-pure nebivolol (Ia) and/or (Ib) is obtained through individual coupling of the 4 separate, enantiomerically-pure intermediates (IIa-d) to the enantiomerically-pure precursors represented by formula (IIIa-d). The production of said precursors (IIIa-d) proceeds via the two enantiomerically-pure ketone derivatives (IVa) and (IVb).

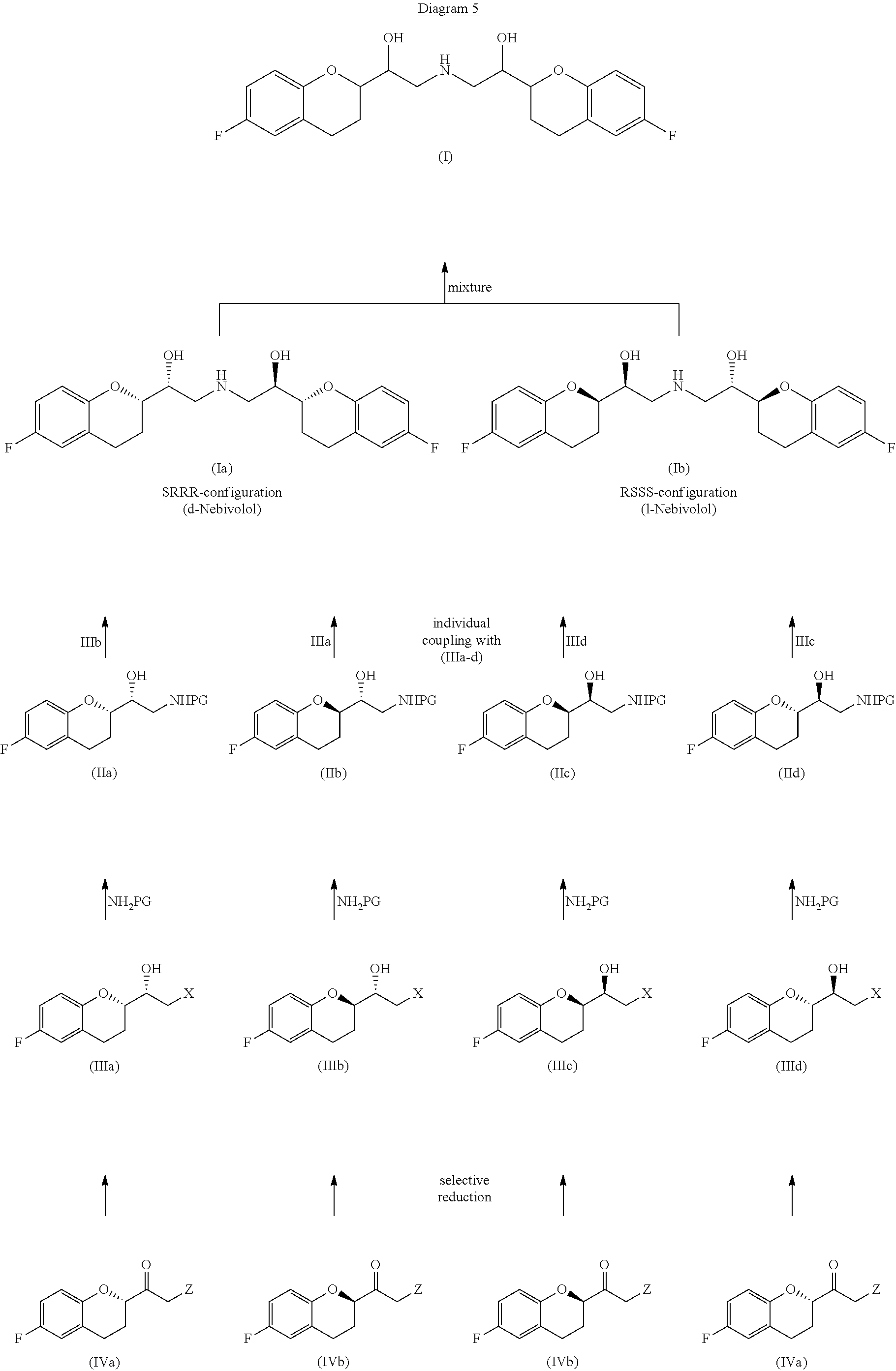

PG in (IIa-d) denotes a hydrogen atom or an amine protection group in each case. Group X in (IIIa-d) represents a halogen atom, a hydroxyl function, an acyl group, an alkylsulfonyloxy group or an arylsulfonyloxy group. Likewise, group Z in (IVa,b) represents a halogen atom, a hydroxyl function, an acyl group, an alkylsulfonyloxy group or an arylsulfonyloxy group.

Compounds (IIa-d) and (IIIa-d) are the key intermediates for the synthesis of racemic nebivolol. Another object of the present invention is to provide methods for producing intermediates (IIa-d) and precursors (IIIa-d) thereof, whereby the methods allow for selective synthesis of said individual isomers at high chemical purity and, mainly, at high optical purity (>98%). Under these conditions, it is possible to forego intermediary purification steps. What is necessary, if applicable, is a recrystallisation of the compounds represented by formula (Ia) and/or (Ib), such that the process for producing nebivolol is both extremely efficient and economical.

Depending on the structure of the target molecule, nebivolol can be prepared very efficiently and selectively from enantiomerically-pure chromanketones IV having a suitable leaving group, e.g. Z=hydroxyl, chlorine, bromine, iodine, mesylate or tosylate.

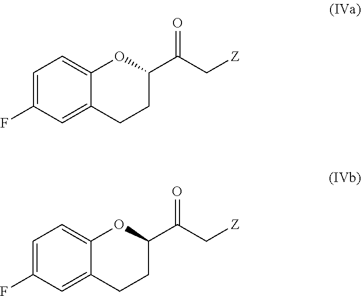

Stereoselective reduction of said ketones results in the corresponding diastereomeric alcohols (IIIa-d), which are precursors of nebivolol. As a prerequisite, it must be feasible to selectively obtain the diastereomeric alcohols at very high purity. According to the invention, the alcohols (IIIa-d) are obtained without purification from the compounds represented by formula (IVa,b) at diastereomeric excess values of >98%, preferably at a diastereomeric excess of >99%, particularly preferred at a diastereomeric excess of >99.5%.

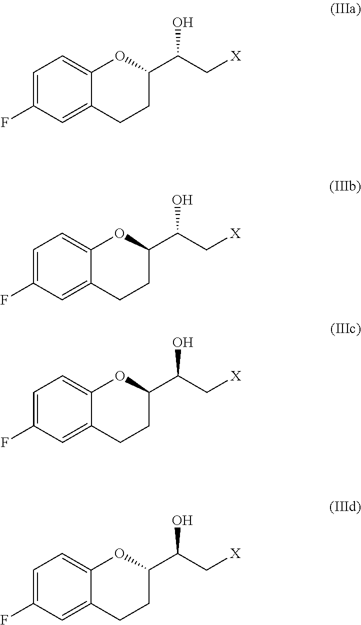

Conceivable reduction agents are chiral complex hydrides, chiral boron compounds (e.g. the chiral borane, CBS, developed by Corey), chiral catalysts for transfer hydrogenations, and catalytic hydrogenations, as well as enzymes.

Surprisingly, it was found that very stereoselective reduction of chroman ketones (IVa) and (IVb) through enzymes is feasible. Enzymatic processes for the preparation of enantiomerically-pure alcohols (IIIa-d) have not been described previously. Selective enzymatic reduction of ketones to form chiral secondary alcohols in the scope of the synthesis of chiral building blocks, as such, is known and can be carried out through alcohol dehydrogenases and ketoreductases (each with and without cofactor regeneration). Said enzymatic methods are also very well-suited for industrial syntheses, since they usually allow very high turnover and very high selectivity to be attained concurrently. The enzymatic reduction of α-chloroketones, in particular, has been investigated extensively. The resulting halohydrins can be coupled to amines either directly or through an indirect route involving the corresponding epoxides. Applied to nebivolol, this means that the target substance can be isolated in well defined form based on the enantiomerically-pure intermediates.

In an enzymatic screening of alcohol dehydrogenases that are commercially available or available to the applicant, it was found that the precursor alcohols (IIIa-d) can be formed through multiple routes of enzymatic means at high selectivity. Many alcohol dehydrogenases possess pronounced R- or S-selectivity with regard to the newly formed centre of asymmetry.

Enzymatic reduction can be used according to the invention to convert the chromane ketones represented by formula (IV a, b) into alcohols represented by formulas III a-d with a quantitative yield and a diastereomeric or enantiomeric purity of >99% in each case. X preferably represents chlorine.

The present invention therefore also relates to a method of enzymatic reduction of chroman ketones represented by formula (IV), whereby X=halogen atom, hydroxyl group or alkyl- or arylsulfonyl groups obtained therefrom, to form enantiomerically-pure compounds represented by general formula (III),

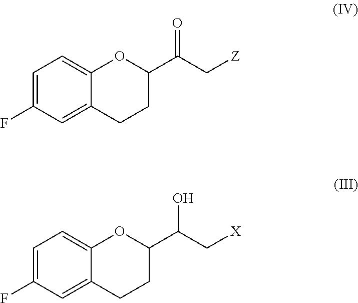

whereby Z represents either a halogen atom, a hydroxyl group or alkyl- or arylsulfonyloxy groups obtained therefrom. Compounds represented by general formula (IV) can be produced easily in racemic or enantiomerically-pure form according to the prior art.

In general, the enzymatic reactions can be carried out in aqueous buffer solutions at room temperature. Alcohol dehydrogenases or ketoreductases can be used as enzymes, whereby the reduction can optionally be carried out to be associated with cofactor regeneration. In this context, the enzymes can be used in isolated or in immobilised form or can just as well be provided through recombinant whole cell systems.

The enzymatic reductions are carried out through in-process controls such that the stereoselectivities are as high as possible, if applicable through premature termination of the reaction at incomplete turnover. The target value, which is attainable according to the invention, is an ee value of >99% of the desired enantiomer.

After separation of the enzyme and/or cells through filtration or centrifugation, the aqueous solution is extracted with organic solvents. In this context, water-immiscible solvents such as methyl-tert.-butylether or ethyl acetate are preferred. After drying of the organic phases, removal of the solvent through distillation yields the enantiomerically-pure alcohols as raw product at such high purity that further purification can be foregone and the products can be converted further directly.

Moreover, the present invention relates to the provision of a method for direct conversion of chiral alcohols (III) into the enantiomerically-pure compounds represented by formula (II),

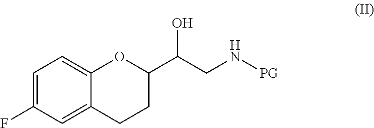

whereby PG represents either an H atom or a protection group (preferably benzyl). The intermediates represented by formula (IIa-d) are obtained from the compounds represented by formula (IIIa-d) at enantiomeric excess values of >98%, preferably at an enantiomeric excess of >99%, particularly preferred at an enantiomeric excess of >99.5% without purification. The conversion proceeds, e.g., through direct coupling to the amine or via the indirect route using the corresponding epoxides in aprotic solvents, such as, e.g., THF or dioxane, under reflux conditions. Direct conversion with benzylamine producing yields of approx. 85-90% without any loss in diastereomeric or enantiomeric purity is preferred. Educts that are not converted can be removed from the raw product through common extraction processes such as washing of the crystalline raw products. Purification of the raw products proved to be unnecessary.

Moreover, the present invention relates to the conversion of compounds represented by formula (II) to d- and/or l-nebivolol. For this purpose, aminoalcohols (II a-d) are cross-coupled to alcohols (III a-d) while exposed to an inorganic base such as potassium carbonate in an aprotic solvent such as THF or dioxane, namely (IIa) to (IIIb) or (IIb) to (IIIa) as well as (IIc) to (IIId) or (IId) to (IIIc). If applicable, a protection group that may be present on the nitrogen is removed, for example a benzyl group through hydrogenation by means of 5% Pd/C in a solvent mixture, e.g. ethanol/glacial acetic acid, typically at approx. 50° C. and a pressure of approx. 3 bar. D-Nebivolol (Ia) and l-nebivolol (Ib) are obtained without prior purification at enantiomeric excess values of >98%, preferably at an enantiomeric excess of >99%, particularly preferred at an enantiomeric excess of >99.5%. Racemic nebivolol is obtained through mixing d-nebivolol (Ia) and 1-nebivolol (Ib).

### EXAMPLES

The following examples illustrate the invention without limiting the scope of the invention to the conditions and substances described specifically.

Preparation of the Buffer Solution for the Enzymatic Reduction:

Dissolve triethanolamine (4 g; 26.5 mmol) in water (215 ml). Adjust the pH of the solution, while stirring, to pH6.99 using 36% HCl (2.3 g). Add ZnCl₂ (0.057 g) and fill up to 270 ml. Then add glycerol (37.5 g) and mix well.

Conducting the Enzymatic Reduction:

Place isopropanol (20 g) in a flask and chill with ice to 0-5° C. Add β-NAD (10 mg) and then add pre-chilled buffer solution (10 ml). Subsequently, add 50 mmol of the chloroketone at 0° C. to the reaction mixture and finally add 6,000 units (S)- or (R)-selective alcohol dehydrogenase. Warm up the sample to 20-25° C. and stir for 24 h. After conversion is complete, centrifuge the reaction solution and extract with ethyl acetate (2×10 ml) after separating the phases. Wash the organic phases with sat. NaCl solution (20 ml) and then dry over Na₂SO₄. The raw product is obtained through removal of the solvent by distillation in a vacuum.

### Example 1

### Preparation of (S)-2-chloro-1-((R)-6-fluoro-3,4-dihydro-2H-chromen-2-yl)-ethanol

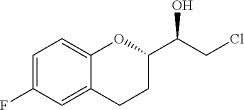 [SMILES: O[C@@H](CCl)[C@@H]1CCc2cc(F)ccc2O1]

1-(2S)-6-fluorochroman-2-yl-2-chloroethan-1-one and (R)-selective alcohol dehydrogenase were used in accordance with the specifications provided above to obtain 11.42 g (99% of theoretical yield) (S)-2-chloro-1-((R)-6-fluoro-3,4-dihydro-2H-chromen-2-yl)-ethanol (d.e. 99.7%).

LC-MS: m/z=230.232 (MH⁺, 100%)

### Example 2

### Preparation of (R)-2-chloro-1-((R)-6-fluoro-3,4-dihydro-2H-chromen-2-yl)-ethanol

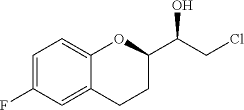 [SMILES: O[C@@H](CCl)[C@H]1CCc2cc(F)ccc2O1]

In analogy to example 1,1-(2R)-6-fluorochroman-2-yl-2-chloroethan-1-one and
(R)-selective alcohol dehydrogenase were used to obtain 11.07 g (96% of theoretical yield) (R)-2-chloro-1-((R)-6-fluoro-3,4-dihydro-2H-chromen-2-yl)-ethanol (d.e. 99.4%).

LC-MS: m/z=230.232 (MH⁺, 100%)

### Example 3

### Preparation of (R)-2-chloro-1-((S)-6-fluoro-3,4-dihydro-2H-chromen-2-yl)-ethanol

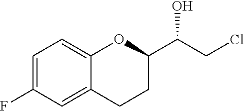 [SMILES: O[C@H](CCl)[C@H]1CCc2cc(F)ccc2O1]

In analogy to example 1,1-(2R)-6-fluorochroman-2-yl-2-chloroethan-1-one and (S)-selective alcohol dehydrogenase were used to obtain 11.42 g (99% of theoretical yield) (R)-2-chloro-1-((S)-6-fluoro-3,4-dihydro-2H-chromen-2-yl)-ethanol (d.e. 99.8%).

LC-MS: m/z=230.232 (MH⁺, 100%)

### Example 4

### Preparation of (S)-2-chloro-1-((S)-6-fluoro-3,4-dihydro-2H-chromen-2-yl)-ethanol

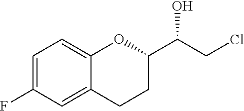 [SMILES: O[C@H](CCl)[C@@H]1CCc2cc(F)ccc2O1]

In analogy to example 1,1-(2S)-6-fluorochroman-2-yl-2-chloroethan-1-one and (S)-selective alcohol dehydrogenase were used to obtain 10.72 g (93% of theoretical yield) (R)-2-chloro-1-((S)-6-fluoro-3,4-dihydro-2H-chromen-2-yl)-ethanol (d.e. 99.5%).

LC-MS: m/z=230.232 (MH⁺, 100%)

### Example 5

### Preparation of (S)-2-benzylamino-1-((R)-6-fluoro-3,4-dihydro-2H-chromen-2-yl)-ethanol

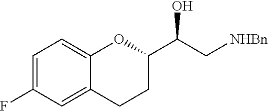

Benzylamine (22 mmol, 2.14 g) was added to a solution of (S)-2-chloro-1-((R)-6-fluoro-3,4-dihydro-2H-chromen-2-yl)-ethanol (10 mmol, 2.307 g) in THF (100 ml) and the resulting mixture was heated and refluxed for 20 h. Subsequently, the solvent was removed through distillation in a vacuum. The remaining residue was then dispersed in tert. butylmethylether (40 ml) and the resulting solid was then suction cleaned. After washing with MTBE (20 ml) and drying in a vacuum, (S)-2-benzylamino-1-((R)-6-fluoro-3,4-dihydro-2H-chromen-2-yl)-ethanol (2.74 g, 91% of theoretical yield), a lightly yellow powder, was obtained at a purity of 98.5% (by HPLC) and having an ee value of 99.6%.

LC-MS: m/z=302 (MH⁺, 100%)

### Example 6

### Preparation of (R)-2-benzylamino-1-((R)-6-fluoro-3,4-dihydro-2H-chromen-2-yl)-ethanol

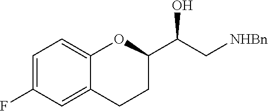

In accordance with the procedure provided in example 5, (R)-2-chloro-1-((R)-6-fluoro-3,4-dihydro-2H-chromen-2-yl)-ethanol (10 mmol, 2.307 g) was reacted with benzylamine to obtain (R)-2-benzylamino-1-((R)-6-fluoro-3,4-dihydro-2H-chromen-2-yl)-ethanol (2.56 g, 85% of theoretical yield) at a purity of 99.2% (by HPLC) and having an ee value of 99.4%.

LC-MS: m/z=302 (MH⁺, 100%)

### Example 7

### Preparation of (R)-2-benzylamino-1-((S)-6-fluoro-3,4-dihydro-2H-chromen-2-yl)-ethanol

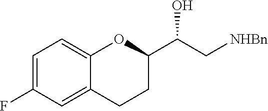

In accordance with the procedure provided in example 5, (R)-2-chloro-1-((S)-6-fluoro-3,4-dihydro-2H-chromen-2-yl)-ethanol (10 mmol, 2.307 g) was reacted with benzylamine to obtain (R)-2-benzylamino-1-((S)-6-fluoro-3,4-dihydro-2H-chromen-2-yl)-ethanol (2.47 g, 82% of theoretical yield) at a purity of 98.8% (by HPLC) and having an ee value of 99.7%.

LC-MS: m/z=302 (MH⁺, 100%)

### Example 8

### Preparation of (S)-2-benzylamino-1-((S)-6-fluoro-3,4-dihydro-2H-chromen-2-yl)-ethanol

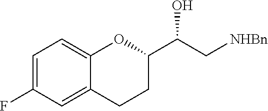

In accordance with the procedure provided in example 5, (S)-2-chloro-1-((S)-6-fluoro-3,4-dihydro-2H-chromen-2-yl)-ethanol (10 mmol, 2.307 g) was reacted with benzylamine to obtain (S)-2-benzylamino-1-((S)-6-fluoro-3,4-dihydro-2H-chromen-2-yl)-ethanol (2.80 g, 93% of theoretical yield) at a purity of 98.6% (by HPLC) and having an ee value of 99.5%.

LC-MS: m/z=302 (MH⁺, 100%)

### Example 9

### Production of Benzylated d-Nebivolol

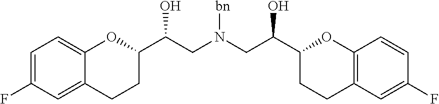

(R)-2-chloro-1-((R)-6-fluoro-3,4-dihydro-2H-chromen-2-yl)-ethanol (10 mmol, 2.31 g) and K₂CO₃ (10 mmol) were added to a solution of (S)-2-benzylamino-1-((R)-6-fluoro-3,4-dihydro-2H-chromen-2-yl)-ethanol (10 mmol, 3.014 g) in dioxane (50 ml). The mixture was heated and refluxed for 24 h. After hot filtration, the solvent was removed through distillation in a vacuum and the residue was dispersed in ethyl acetate (20 ml). The solid was separated by filtration and washed thoroughly with ethyl acetate. After drying in a vacuum, 3.62 g of product (73% of theoretical yield) of a purity of 98.6% (by HPLC) and having an ee. value of 99.6% were obtained.

LC-MS: m/z=496 (MH⁺, 100%)

### Example 10

### Production of Benzylated l-Nebivolol

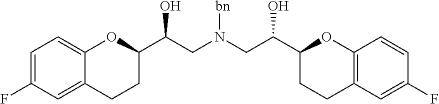

In accordance with the procedure provided in example 9, (R)-2-benzylamino-1-((S)-6-fluoro-3,4-dihydro-2H-chromen-2-yl)-ethanol (10 mmol, 3.014 g) and (S)-2-chloro-1-((S)-6-fluoro-3,4-dihydro-2H-chromen-2-yl)-ethanol (10 mmol, 2.307 g) were used to obtain the product (3.42 g, 73% of theoretical yield) at a purity of 99.1% (by HPLC) and having an ee. value of 99.5%.

LC-MS: m/z=496 (MH⁺, 100%)

### Example 11

### Production of d-Nebivolol

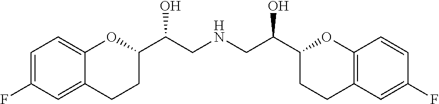 [SMILES: O[C@H](CNC[C@@H](O)[C@H]1CCc2cc(F)ccc2O1)[C@@H]1CCc2cc(F)ccc2O1]

Benzylated d-nebivolol from example 9 (4.96 g; 10 mmol) was debenzylated in ethanol (50 ml) and glacial acetic acid (5 ml) using 5% Pd/C at a pressure of 2-3 bar and a temperature of 40-50° C. The reaction mixture was filtered through Celite and the filtrate was concentrated in a vacuum. Traces of glacial acetic acid were removed through subsequent evaporation in the presence of 2×25 ml ethanol. Ethyl acetate (50 ml) was added to the resulting raw material, this was filtered, and subsequently heated and refluxed. After cooling and filtration, d-nebivolol (3.98 g; 98% of theoretical yield) was obtained at a chemical purity of 99.5% and having an enantiomeric excess of 99.6%.

LC-MS: m/z=406 (MH⁺, 100%)

### Example 12

### Production of l-Nebivolol

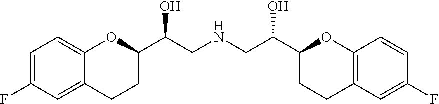 [SMILES: O[C@@H](CNC[C@H](O)[C@H]1CCc2cc(F)ccc2O1)[C@@H]1CCc2cc(F)ccc2O1]

In accordance with the procedure provided in example 11, 4.96 g (10 mmol) benzylated l-nebivolol from example 10 were used to obtain l-nebivolol (3.86 g; 95% of theoretical yield) at a chemical purity of 99.4% and having an enantiomeric excess of 99.5%.

LC-MS: m/z=406 (MH⁺, 100%)

1. Method for synthesis of racemic nebivolol represented by formula (I) 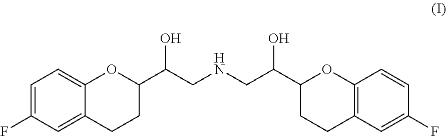 [SMILES: OC(CNCC(O)C1CCc2cc(F)ccc2O1)C1CCc2cc(F)ccc2O1]
from the enantiomerically-pure compounds represented by formula IVa and IVb 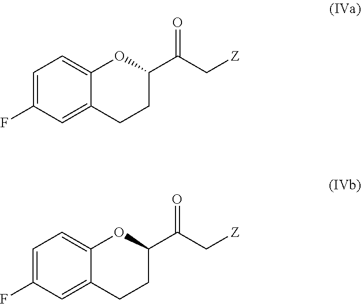
whereby racemic nebivolol is obtained through mixing the enantiomerically-pure compounds d-nebivolol (Ia) and l-nebivolol (Ib), 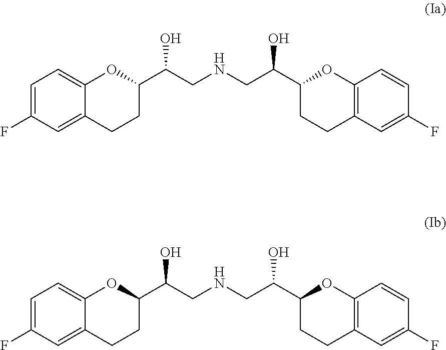 [SMILES: O[C@H](CNC[C@@H](O)[C@H]1CCc2cc(F)ccc2O1)[C@@H]1CCc2cc(F)ccc2O1 ; O[C@@H](CNC[C@H](O)[C@H]1CCc2cc(F)ccc2O1)[C@@H]1CCc2cc(F)ccc2O1]
the compounds d-nebivolol (Ia) and l-nebivolol (Ib) are synthesised independent of each other as enantiomerically-pure compounds through producing (Ia) and (Ib) each through individual coupling of the 4 enantiomerically-pure intermediates represented by formula (IIa-d) to the corresponding precursors represented by formula (IIIa-d); 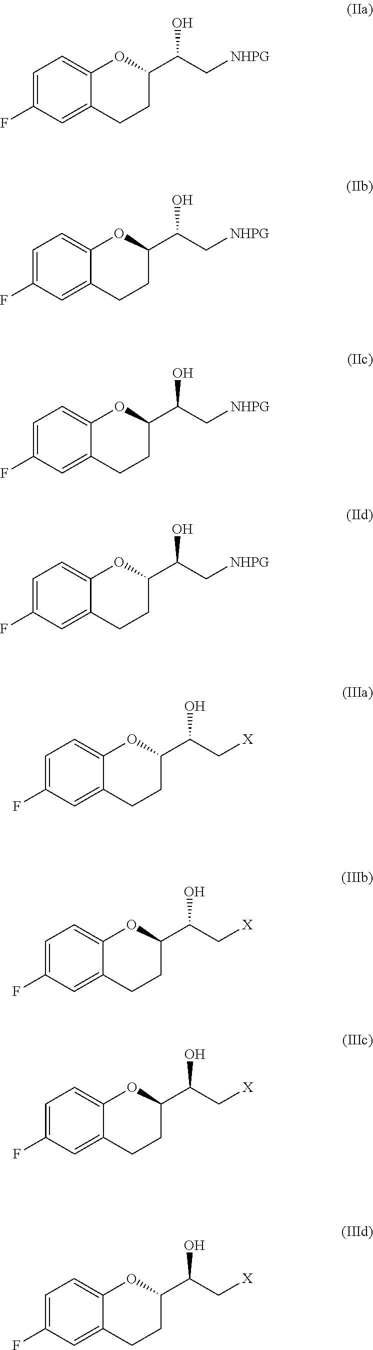
whereby d-nebivolol (Ia) is obtained through coupling (IIa) to (IIIb) or (IIb) to (IIIa) and l-nebivolol (Ib) is obtained through coupling (IIc) to (IIId) or (IId) to (IIIc), and PG in the intermediates represented by formula (IIa-d) is a hydrogen atom or an amine protection group, and X in the precursors represented by formula (IIIa-d) is a halogen atom, a hydroxyl group, an acyl group, an alkylsulfonyloxy group or an arylsulfonyloxy group,
the intermediates represented by formula (IIa-d) are formed individually from the respective direct precursors represented by formula (IIIa-d), whereby intermediate (IIa) is formed from (Ma), intermediate (IIb) is formed from (IIIb), intermediate (IIe) is formed from (IIIc), and intermediate (IId) is formed from (IIId);
the precursors represented by formula (IIIa-d) are formed specifically through reduction of the enantiomerically-pure ketone precursors represented by formula (IVa,b), whereby the precursors represented by formula (IIIa) and (IIId) originate from the ketone precursor represented by formula (IVa), and the precursors represented by formula (IIIb) and (IIIc) originate from the ketone precursor represented by formula (IVb), and Z in the ketone precursors (IVa,b) is a halogen atom, a hydroxyl function, an acyl group, an alkylsulfonyloxy group or an arylsulfonyloxy group.

2. Method according to claim 1, wherein the intermediates (IIa-d) are formed from the enantiomerically-pure precursors (IIIa-d) through diastereoselective reduction using a highly selective chiral reduction agent.

3. Method according to claim 1, wherein a reduction agent selected from chiral complex hydrides, chiral boron compounds, chiral catalysts for transfer hydrogenations and catalytic hydrogenations, and enzymes is used for reduction of compounds (IVa,b).

4. Method according to claim 3, wherein enzymes are used for reduction of compounds (IVa,b).

5. Method according to claim 4, wherein alcohol dehydrogenases and ketoreductases are used with or without cofactor regeneration for reduction of compounds (IVa,b).

6. Method according to claim 1, wherein the intermediates represented by formula (IIa-d) are formed from the precursors represented by formula (IIIa-d) through reaction with ammonia or an arylamine, preferably benzylamine.

7. Method for the reduction of chroman ketones represented by formula (IV), where X=halogen atom, hydroxyl group or alkyl- or arylsulfonyloxy groups obtained therefrom, to the enantiomerically-pure compounds represented by general formula (III) 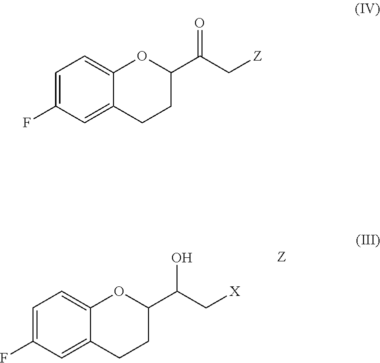
wherein the reduction proceeds by enzymatic means through alcohol dehydrogenases and/or ketoreduktases with or without cofactor regeneration.

8. Method for synthesis of nebivolol enantiomers represented by formula (I) 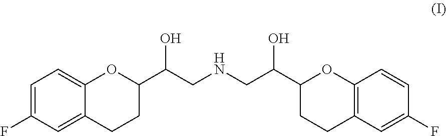 [SMILES: OC(CNCC(O)C1CCc2cc(F)ccc2O1)C1CCc2cc(F)ccc2O1]
from the enantiomerically-pure compounds represented by formula IVa and IVb; 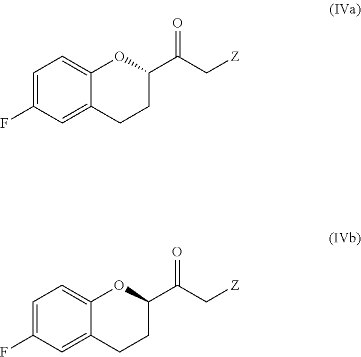
whereby the nebivolol enantiomers are synthesised through individual coupling of the 4 enantiomerically-pure intermediates represented by formula (IIa-d) to the corresponding precursors represented by formula (IIIa-d); 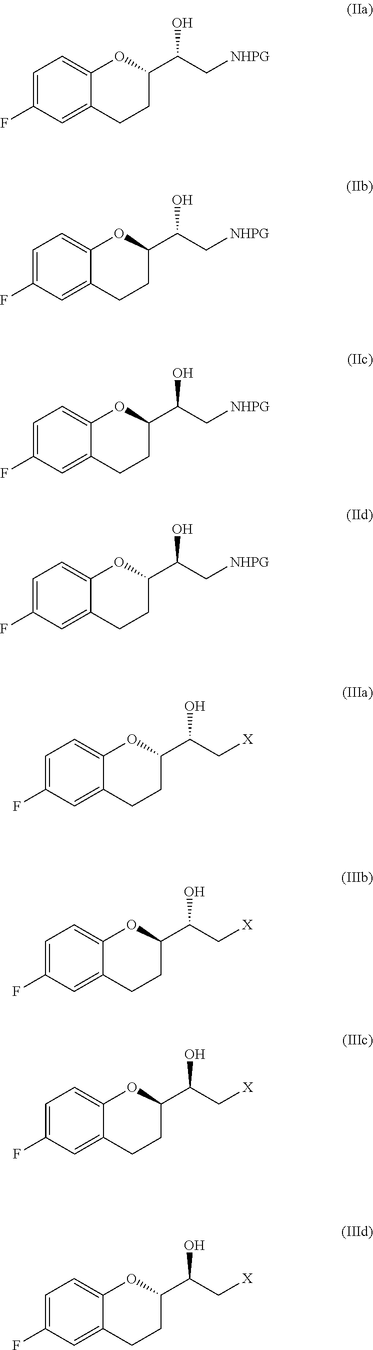
whereby PG in the intermediates represented by formula (IIa-d) is a hydrogen atom or an amine protection group, and X in the precursors represented by formula (IIIa-d) is a halogen atom, a hydroxyl group, an acyl group, an alkylsulfonyloxy group or an arylsulfonyloxy group;
the intermediates represented by formula (IIa-d) are formed individually from the respective direct precursors represented by formula (IIIa-d), whereby intermediate (IIa) is formed from (IIIa), intermediate (IIb) is formed from (IIIb), intermediate (IIc) is formed from (IIIc), and intermediate (IId) is formed from (IIId);
the precursors represented by formula (IIIa-d) are formed specifically through reduction of the enantiomerically-pure ketone precursors represented by formula (IVa,b), whereby the precursors represented by formula (IIIa) and (IIId) originate from the ketone precursor represented by formula (IVa), and the precursors represented by formula (IIIb) and (IIIc) originate from the ketone precursor represented by formula (IVb), and Z in the ketone precursors (IVa,b) is a halogen atom, a hydroxyl function, an acyl group, an alkylsulfonyloxy group or an arylsulfonyloxy group.
